(19) United States
(12) Patent Application Publication
(10) Pub. No.: US 2025/0263460 A1
(43) Pub. Date: Aug. 21, 2025

(54) RAAV-CONE OPSIN COMPOSITIONS AND METHODS FOR TREATING BLUE CONE MONOCHROMACY AND COLOR BLINDNESS

(71) Applicant: West Virginia University Board of Governors on behalf of West Virginia University, Morgantown, WV (US)

(72) Inventor: Wen-Tao Deng, Morgantown, WV (US)

(73) Assignee: West Virginia University Board of Governors on behalf of West Virginia University, Morgantown, WV (US)

(21) Appl. No.: 18/858,629

(22) PCT Filed: Apr. 21, 2023
(86) PCT No.: PCT/US2023/066041

§ 371 Date: Oct. 21, 2024

Related U.S. Application Data
(60) Provisional application No. 63/363,420, filed on Apr. 22, 2022.

(51) Int. Cl. C07K 14/705; A61K 38/00; A61K 48/00; C12N 15/86

(52) U.S. Cl. CPC C07K 14/705; A61K 48/0058; C12N 15/86; A61K 38/00; C12N 2750/14143; C12N 2830/008

(57) ABSTRACT

Viral vector compositions are provided comprising polynucleotide sequences that express one or more biologically-active mammalian cone opsin proteins, under the control of a cone photoreceptor cell specific promoter, and a pharmaceutically acceptable carrier. Methods for use of these viral vector compositions are disclosed for in preventing, treating, and/or ameliorating a mammalian subject having a disease associated with cone monochromacies, such as blue cone monochromacy (BCM). Specifically, these compositions are disclosed which are useful in methods for treating or ameliorating symptoms of blue cone monochromacy caused by mis-sense point mutations, such as C203R, in the red and/or green cone opsin genes (OPN1LW/OPN1MW).

### CROSS REFERENCE TO RELATED APPLICATION

This utility non-provisional patent application claims the benefit of priority to U.S. Provisional Patent Application Ser. No. 63/363,420, filed Apr. 22, 2022. The entire contents of U.S. Provisional Patent Application Ser. No. 63/363,420 are incorporated by reference into this utility non-provisional patent application as if fully written herein.

### STATEMENT REGARDING FEDERALLY SPONSORED RESEARCH OR DEVELOPMENT

This invention was made with government support under Grant No. R01 EY030056 awarded by the National Institutes of Health. The government has certain rights in the invention.

### SEQUENCE LISTING STATEMENT

The Sequence Listing (WIPO Standard ST.26 XML file) submitted herewith and incorporated by reference in its entirety by Applicant does not go beyond the disclosure of the International Application as filed. The computer readable Sequence Listing WIPO Standard ST.26 XML file (named “0074539-000144.xml”; date of creation: Apr. 20, 2023, and 6,404 bytes in size) and the paper form Sequence Listing provided in the specification of the international application as filed are the same. End of Statement.

### BACKGROUND OF THE INVENTION

### 1. Field of the Invention

The present invention relates generally to the fields of molecular biology and virology, and in particular, to methods for using recombinant adeno-associated virus (rAAV) compositions that express at least a first nucleic acid segment encoding at least a first therapeutic gene product, and particularly those products useful in the prevention, treatment, or amelioration of one or more symptoms of diseases, disorders, trauma, injury, or dysfunction of the mammalian eye. In particular embodiments, the invention provides compositions including rAAV vectors that express a biologically-functional cone opsin peptide, polypeptide, or protein for use in one or more investigative, diagnostic and/or therapeutic regimens, including, for example, the treatment of one or more disorders or diseases of the mammalian eye, and in particular, for treating congenital retinal blindness including, retinal dystrophy such as blue cone monochromacy (BCM) in humans caused by mis-sense point mutations, such as C203R, in the OPN1LW/OPN1MW.

Also provided are methods for preparing rAAV vector-based medicaments for use in viral vector-based gene therapies, including, for example rAAV-OPN1LW or rAAV-OPN1MW vectors for treating or ameliorating one or more symptoms of cone opsin deficiency in humans.

### 2. Background Art

In the human retina, L- and M-cones constitute about 95% of the total cone population and are primarily concentrated in the central macula responsible for our daylight, color, and fine spatial vision¹⁻⁴. X-linked retinal diseases resulting from mutations in the L-(long-wavelength,OPN1LW) and M-(middle-wavelength, OPN1MW) opsin genes are associated with a wide range of visual defects including red/green color vision deficiency, blue cone monochromacy, X-linked cone dystrophy/dysfunction, and high myopia with abnormal cone function⁵⁻¹⁹.

Blue cone monochromacy (BCM) is an X-linked congenital vision disorder with severe cone dysfunction due to the mutations in both long (OPN1LW) and medium (OPN1MW) wavelength-sensitive cone opsins¹. BCM patients display markedly reduced central vision, severely impaired color vision, photophobia (bright light sensitivity), and congenital nystagmus?. In humans, L-opsin (OPN1LW) and M-opsin (OPN1MW) genes are tandemly arrayed on the X-chromosome in a head to tail arrangement with a single L-opsin gene in the 5′ position followed by one or more M-opsin genes.²⁰ Expression of both OPN1LW and OPN1MW is regulated by specific proximal promoters and a single upstream locus control region (LCR), ensuring that only one opsin gene is expressed in a single cone photoreceptor.²¹⁻²³ It has been shown that only the first two genes in the cluster are normally expressed.^{22.24} The L and M cone opsins are highly homologous and share 96% amino acid identity. This sequence homology and close genomic proximity predispose the two pigment genes to homologous recombination resulting in gene deletions, duplications, or fusion genes that consist of portions of both red and green pigment genes.^{10,25, 26} Mutations in the locus control region or harmful mutations in both of the genes resulting in absence of both functional cone pigments are the genetic cause of blue cone monochromacy (BCM)^{9, 16, 21, 27-29}

The two most common causes of BCM are deletions encompassing the LCR, or the presence of deleterious C203R missense mutation either in a single OPN1LW/MW hybrid gene or in multiple OPN1LW/MW genes.^{16,21,27} BCM affects 1 in 100,000 individuals and BCM patients who must rely on remaining preserved S-cones and rod photoreceptors display severely impaired color discrimination from birth, and they typically suffer from reduced visual acuity that may progress to 20/200, myopia, pendular nystagmus, and photoaversion.^{14, 30}

C203R accounts for about 60% of BCM patients¹⁶. C203R is highly conserved in all visual opsins, with the corresponding mutation in rhodopsin (C187Y) causing early and severe adRP³¹. Cysteines at residues 126 and 203 of cone opsins form a disulfide bond between the third transmembrane helix and the second extracellular loop, thus the mutation disrupts the proper tertiary structure of cone opsin. C203R opsin expressed in cell culture is improperly folded and retained in the ER²⁷.

There has been a long history of investigation of the clinical, electrophysiological, and psychophysical aspects in BCM.^{5,32,33} Recently, studies using Adaptive Optics Scanning Laser Ophthalmoscopy (AOSLO) showed that BCM patients have a disrupted foveal cone mosaic with reduced numbers of cones. While remaining cones have detectable outer segments, they are significantly shortened with fewer disk membranes. The identification of residual cone structure in the BCM fovea suggests that cones can survive without opsin albeit in reduced numbers, making them a potentially viable target for gene therapy.³⁴⁻³⁶

Those persons of ordinary skill in the art appreciate the deficiencies in the technology of the background art. BCM patients with deletions in the LCR which cause abolished expression of L-and M-opsins can be potentially treated with gene replacement therapy as we have recently demonstrated in the Opn1mw^{-/-} and Opn1mw^{-/-} Opn1sw^{-/-} mouse models of BCM^{37, 38, 39}. See U.S. Pat. No. 10,533,187 B2. However, there are no effective prophylactics or therapeutics available to prevent or treat BCM patients carrying point mis-sense mutations such as C203R, account about 60% total BCM cases. Because L/M-opsin genes are X-linked, affected males express only the mutant copy, while in female carriers, due to X-linked inactivation, about half of their cones express a wild-type copy while the other half cones express the mutant copy. Therefore, it is not known whether currently identified cone opsin point mutants have a negative gain of function effect on cone photoreceptors. That raises the question of if gene replacement therapy would work for cone opsin mis-sense mutations presented in BCM patients.

### SUMMARY OF THE INVENTION

Provided are viral vector compositions comprising polynucleotide sequences that express one or more biologically-active mammalian cone opsin proteins, under the control of a cone photoreceptor cell specific promoter, and a pharmaceutically acceptable carrier. Also disclosed are methods for their use in preventing, treating, and/or ameliorating a mammalian subject having a disease associated with cone monochromacies, such as blue cone monochromacy (BCM). Specifically, these and other compositions are disclosed with are useful in methods for treating or ameliorating symptoms of blue cone monochromacy caused by mis-sense point mutations, such as C203R, in the red and/or green cone opsin genes (OPN1LW/OPN1MW).

In certain embodiments of this invention, a composition comprising rAAV vectors that express a biologically-functional cone opsin peptide, polypeptide, or protein, and a pharmaceutically acceptable carrier, are provided. The composition includes wherein said rAAV vector expresses either rAAV-OPN1LW or rAAV-OPN1MW.

In other embodiments of this invention, a method is provided for treating a patient having an eye disease, disorder, trauma, injury, or dysfunction comprising administering to a patient a therapeutically effective amount of a composition comprising a rAAV vector that express a biologically-functional cone opsin peptide, polypeptide, or protein, and a pharmaceutically acceptable carrier. This method includes wherein said rAAV vector is either rAAV-OPN1LW or rAAV-OPN1MW. The method may include wherein said disorder or disease is of a mammalian eye, and is a congenital retinal blindness. This method includes wherein said congentital retinal blindness is selected from the group of retinal dystrophy such as cone opsin deficiency and blue cone monochromacy (BCM) in humans caused by mis-sense point mutations, such as C203R, in the OPN1LW/OPN1MW.

In yet another embodiment of this invention, a method is provided for preparing a rAAV vector-based composition for use in viral vector-based gene therapies, including, preparing a rAAV-OPN1LW vector or a rAAV-OPN1MW vector driven by a cone specific promoter PR2.1.

Another embodiment of this invention provides a method of treating a patient having blue cone monochromacy comprising administering to a patient a therapeutically effective amount of a composition gene replacement using rAAV vectors that encode one or more mammalian cone opsins polypeptides for treating a cone photoreceptor function of the patent. This method includes wherein said rAAV vector expresses either rAAV-OPN1LW or rAAV-OPN1MW. This method includes wherein said rAAV vector expresses either rAAV-OPN1LW or rAAV-OPN1MW driven by a cone specific promoter PR2.1.

In certain embodiments of this invention, a viral vector is provided comprising a vector containing a human OPN1MWcDNA driven by cone-specific PR2.1 promoter12.

In certain embodiments of this invention, a vector containing a human OPN1MWcDNA driven by cone-specific PR2.1 promoter is provided.

In another embodiment of this invention, a method is disclosed of treating a patient carrying a C203R mutation wherein cysteine in protein position 203 is mutated to arginine, comprising administering to a patient a therapeutically effective amount of a recombinant adeno-associated viral vector expressing either human L-opsin or M-opsin driven by a cone photoreceptor-cell specific promoter. This method includes wherein said administration of said composition is by injecting said composition into an eye of said patient.

A SEQ ID NO:2 of hOPN1MW and a SEQ ID NO:3 of hOPN1LW are provided.

### BRIEF DESCRIPTION OF THE DRAWINGS

FIG. 1 shows two major causes of BCM: one is caused by large deletions in the locus control region (LCR) which abolishes expression of both OPN1LW and OPN1MW; one is caused by deleterious mis-sense mutation C203R presenting in either a single hybrid OPN1M/LW or in both OPN1M/LW and OPN1MW

FIG. 2 shows genotyping and sequencing result of Opn1mw^{C198R} mice. TGT (C) in wild-type mice is mutated to CGT (R) to create Opn1mw^{C198R} mice. An ApaI restriction site (GGGCCC) was introduced (without changing amino acid sequence) after CGT to facilitate genotyping.

FIG. 3A shows representative retinal whole mounts stained with Peanut agglutinin (PNA) to characterize cone photoreceptor degeneration.

FIG. 3B shows the numbers of PNA positive cells were counted from dorsal and ventral areas of Opn1mw^{C198R}Opn1sw^{-/-} and wild-type mice. Opn1mw^{C198R}Opn1sw^{-/-} mice have similar numbers of viable cones as wildtype mice at 1 month of age, but cones degenerate between 1 and 6 months and stabilize by 6 months of age. N=6 mice (3 Females and 3 males) were used for each group. 4 images (2 from dorsal and 2 from ventral) were taken from each retinas. P<0.05.

FIG. 3C shows cone arrestin (red-top line) and Secretagogin (green-bottom area) staining to characterize cones and cone bipolar in aged Opn1mw^{C198R}Opn1sw^{-/-} mice. Cone arrestin staining showed that cones degenerate with age and cone outer segments are absent in Opn1mw^{C198R}Opn1sw^{-/-} mice. Secretagogin staining showed that cone bipolar cells appeared to be normal up to 12 months of age.

FIG. 4A shows that Opn1mw^{C198R}Opn1sw^{-/-} mice have abolished photopic electroretinal responses (ERG) while maintain normal scotopic ERG; representative middle-wavelength mediated ERG traces from wild-type (grey (1)) and Opn1mw^{C198R}Opn1sw^{-/-} (red (2)) mice.

FIG. 4B shows representative white light mediated ERG traces from wild-type (grey (1)) and Opn1mw^{C198R}Opn1sw^{-/-} (red (2)) mice.

FIG. 4C shows averaged white light b-wave maximum amplitudes at light intensity of 25 cd·s/m² from wild-type and Opn1mw^{C198R}Opn1sw^{-/-} mice.

FIG. 4D shows averaged scotopic a-wave maximum amplitudes at different intensities to show that rod function is normal in Opn1mw^{C198R}Opn1sw^{-/-} mice.

FIG. 5A shows that cone outer segments are absent in Opn1mw^{C198R}Opn1sw^{-/-} mice. Cone outer segments were stained with cone phosphodiesterase α′ (PDE6C) and GNAT2 in Opn1mw^{C198R}Opn1sw^{-/-} and wildtype mice. PDE6α′ and GNAT2 stainings are absent in Opn1mw^{C198R}Opn1sw^{-/-} retinas.

FIG. 5B shows a Western blot analysis. The Western blot shows that expression of Opn1mw^{C198R} mutant opsin was not detected. The faint band near 37 KD was a non-specific band also present in the Opn1mw^{-/-}Opn1sw^{-/-} retinas (labeled as DKO) which have abolished expression of OPN1MW.

FIG. 6 shows real-time PCR results that mRNA levels of Opn1mw^{C198R} mutant in Opn1mw^{C198R}Opn1sw^{-/-} retinas are normal at postal natal 5 (P5) but only ˜50% of wild-type levels at P15 and P30.

FIG. 7A shows gene therapy rescued cone function and structure in Opn1mw^{C198R} Opn1sw^{-/-} mice when treated at 1 month of age and shows representative cone-mediated medium-wavelength ERG traces from wild-type, untreated Opn1mw^{C198R} Opn1sw^{-/-} and AAV5-hOPN1MW-treated at 1 month of age and ERGed at 1 month and 4 months post-injection.

FIG. 7B shows averaged b-wave maximum amplitudes of middle-wavelength mediated ERG responses in Opn1mw^{C198R} Opn1sw^{-/-} untreated, wild-type controls, and Opn1mw^{C198R} Opn1sw^{-/-} treated with AAV5-hOPN1MW at 1 month post-injection (1M+1M) and 4 months post-injection (1M+4M).

FIG. 7C shows immunohistochemistry shows that treatment restored expression and normal localization of cone outer segment specific proteins of GNAT2 and PDC6C.

FIG. 8 shows western blot analysis OPN1M/LW expression in treated Opn1mw^{C198R} Opn1sw^{-/-} eyes injected at 1 month of age and analyzed 1 month and 4 months post-injection. An TUBA 4A antibody was used as loading control.

FIG. 9A shows gene therapy rescued cone function and structure in Opn1mw^{C198R} Opn1sw^{-/-} mice when treated at 3 month of age, and shows representative cone-mediated medium-wavelength ERG traces from wild-type (black waveforms (1)), untreated Opn1mw^{C198R} Opn1sw^{-/-} (red waveforms (2)) and AAV5-hOPN1MW-treated at 3 month of age and ERGed at 1month (blue waveforms (3)) and 4 months (green waveforms (4)) post-injection.

FIG. 9B shows averaged b-wave maximum amplitudes of middle-wavelength mediated ERG responses in Opn1mw^{C198R} Opn1sw^{-/-} untreated, wild-type controls, and Opn1mw^{C198R} Opn1sw^{-/-} treated with AAV5-hOPN1MW at 1month post-injection (3M+1M) and 4 months post-injection (3M+4M).

FIG. 9C shows immunohistochemistry shows that treatment restored expression and normal localization of cone outer segment specific proteins of GNAT2 and PDC6C.

FIG. 10A shows cone-mediated function was maintained for long-term in Opn1mw^{C198R} Opn1sw^{-/-} mice treated at 1 and 3 months and shows averaged b-wave maximum amplitudes of middle-wavelength mediated ERG responses in Opn1mw^{C198R} Opn1sw^{-/-} untreated, wild-type controls, and Opn1mw^{C198R}Opn1sw^{-/-} treated at 1 month and ERGed 10 month post-injection (1M+10M), and treated at 3 month and ERGed 7 month post-injection (3M+7M).

FIG. 10B shows immunohistochemistry shows that treatment restored expression and normal localization of cone outer segment specific proteins of GNAT2 and PDC6C.

FIG. 11A shows that treatment efficacy was significantly diminished when Opn1mw^{C198R} Opn1sw^{-/-} mice were treated at 5 months of age and shows only ˜29% (9 out of 31 mice) of eyes treated at 5 months of age showed any cone rescue above 20 μV, while ˜75% of eyes (22 out of 29) treated at 1 months of age showed cone rescue above 50 μV.

FIG. 11B shows immunohistochemistry shows that although a lot more cones are present by PNA staining, however, only 30-50% of PNA positive cones showed OPN1M/LW staining.

FIG. 11C shows in 5M+1M treated eyes, only ˜50% of OPN1M/LW positive cells showed GNAT2 expression and the staining is much weaker compared to mice treated at 1 month and 3 month.

FIG. 11D shows in 5M+1M treated eyes, only ˜50% of OPN1M/LW positive cells showed PDE6C expression and the staining is weaker compared to mice treated at 1 month and 3 month.

FIG. 12A shows in Opn1mw^{C198R} homozygous female mice, misfolded Opn1mw^{C198R} protein was not detected by immunohistochemistry. While in Opn1mw^{C198R} heterozygous female, the number of M-opsin positive cells are about half of in the wild-type mice. M-opsin was labeled as red and S-opsin was labeled as green.

FIG. 12B shows in Opn1mw^{C198R} mice, there is no M-cone ERG in homozygous Females and hemizygous males, and M-cone ERG is reduced in heterozygous females. The S-cone ERG function is normal in Opn1mw^{C198R} mice. The photopic ERG is also reduced in Opn1mw^{C198R} mice most likely due to loss of S-cone function.

FIG. 13 shows on the top row dorsal and ventral retinas and bottom row shows that cones degenerate in the dorsal retinas of 6 months old Opn1mw^{C198R} mice. Cones were labeled with PNA. There were less PNA positive cells in the dorsal retinas than in the ventral area suggesting cones die gradually with age.

FIG. 14 shows averaged b-wave maximum amplitudes of middle-wavelength mediated ERG responses in Opn1mw^{C198R} untreated mice, Opn1mw^{C198R} mice treated with either AAV5-PR2.1-hOPN1MW.HA or AAV5-PR2.1-hOPN1LW, and isogenic wild-type controls. Opn1mw^{C198R} mice were treated at 1 month of age and ERGed at 1 month post-injection.

FIG. 15 top row (left) and bottom row (left) shows untreated dorsal retina, and top row (right) shows dorsal and ventral retinas treated with AAV-mediated hOPN1MW.HA expression in cone outer segments in both dorsal (bottom middle FIG. 15) and ventral (bottom right FIG. 15) areas of treated Opn1mw^{C198R} mice. HA tag was included in frame at the C-terminal of hOPN1MW.

FIG. 16 shows AAV5-PR2.1-hOPN1MW.HA gene therapy restored cone phosphodiesterase γ′ subunit (PDE6 γ′) expression and subcellular localization in the dorsal retinas of treated Opn1mw^{C198R} mice. In the dorsal retinas of untreated Opn1mw^{C198R} mice, cone outer segments were significantly shortened, and no PDE6 γ′ expression was detected. In contrast, in the AAV5-PR2.1-hOPN1MW.HA treated dorsal retinas, treatment restored normal cone outer segments and PDE6 γ′ expression and localization.

FIG. 17 shows a map of an illustrative vector of a certain embodiment of this invention containing the human OPN1MW cDNA driven by cone-specific PR2.1 promoter.

FIG. 18 shows amino acid sequence alignment of mouse OPN1MW (i.e. mOPN1MW), human OPN1MW (i.e. hOPN1MW), and human OPN1LW (i.e. hOPN1LW). Protein sequences are highly conserved between mouse and human opsins.

### DETAILED DESCRIPTION OF THE INVENTION

As used herein, the term “patient” refers to a member of the animal kingdom, including but not limited to homo sapiens.

As used herein, the term “therapeutically effective amount” refers to that amount of a substance, compound, or composition needed to bring about a desired result, such as for example but not limited to treating a patient.

Our previous study showed that point mis-sense mutation of cone opsins might have a negative gain of function effect on cone photoreceptors⁴⁰. Typically gain of function caused by point mutations cannot be treated with simple gene replacement therapy. However, the present invention demonstrated that gene replacement using rAAV is effective in treating BCM patients with point mutations such as C203R, and maybe other cone opsin point mutations. We demonstrated that rAAV-based genetic constructs that encode one or more therapeutic mammalian cone opsins polypeptides restored cone photoreceptor function and cone outer segment structure in the mouse models resemble human patients carrying C203R mutation.

We used two mouse models in this study. Opn1mw^{C198R} knock-in mice have a C198R point mutation in the mouse M-opsin gene which corresponds to human cone opsin C203R mutation presented in BCM patients. This mouse model was generated by CRISPR/Cas technology. We also crossed Opn1mw^{C198R} mice to Opn1sw^{-/-} background to remove the interference from endogenous S-opsin since most mouse cones co-express both M- and S-opsins. This strain is designated as Opn1mw^{C198R}Opn1sw^{-/-} mice. Both stains of mice have undetectable level of M-cone mediated visual function. Opn1mw^{C198R} Opn1sw^{-/-} mice have significantly shortened or no cone outer segment structure across the entire retina, and Opn1mw^{C198R} mice have significantly shortened or no cone outer segment structure in the dorsal retinas where M-opsin predominately expressed. The retinal function and cone morphology displayed in both strains of mice resemble retinal phenotype displayed in human patients. Interestingly, misfolded Opn1mw^{C198R} protein is not detected in either strain of mice by immunohistochemistry suggesting it is degraded efficiently.

We injected both strains of mice subretinally with AAV vector expressing either human OPN1LW or OPN1MW driven by the cone specific promoter PR2.1. We show that AAV delivered M-opsin localizes specifically in cone outer segments. In addition, gene therapy rescued cone photoreceptor function, and restored cone outer segment structure, and restored expression of other cone outer segment specific proteins. Our study demonstrates that cones expressing misfolded Opn1mw^{C198R} protein remain viable and respond to gene augmentation therapy, thereby providing proof-of-concept for cone function restoration in BCM patients with mis-sense mutations.

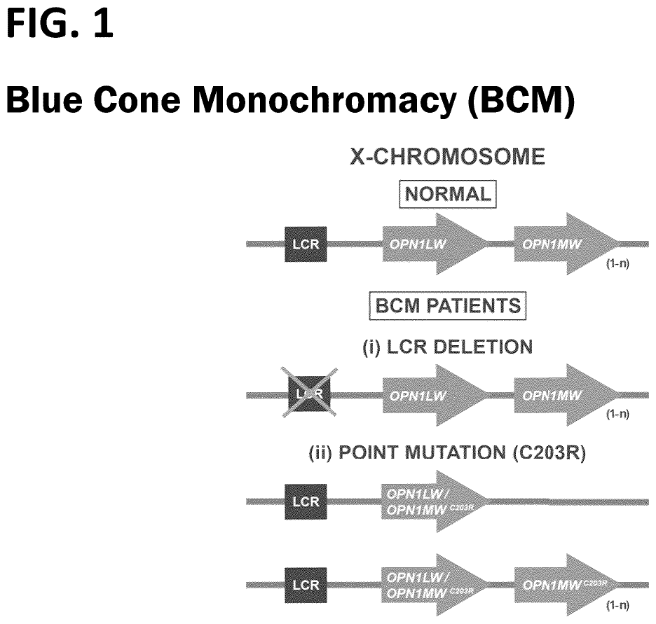

FIG. 1 shows two major causes of BCM: one is caused by large deletions in the locus control region (LCR) which abolishes expression of both OPN1LW and OPN1MW; one is caused by deleterious mis-sense mutation C203R presenting in either a single hybrid OPN1M/LW or in both OPN1M/LW and OPN1MW

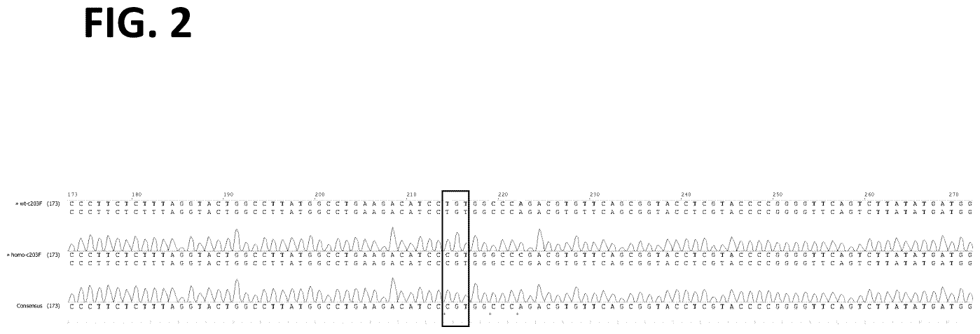

FIG. 2 shows genotyping and sequencing result of Opn1mw^{C198R} mice. TGT (C) in wild-type mice is mutated to CGT (R) to create Opn1mw^{C198R} mice. An ApaI restriction site (GGGCCC) was introduced (without changing amino acid sequence) after CGT to facilitate genotyping.

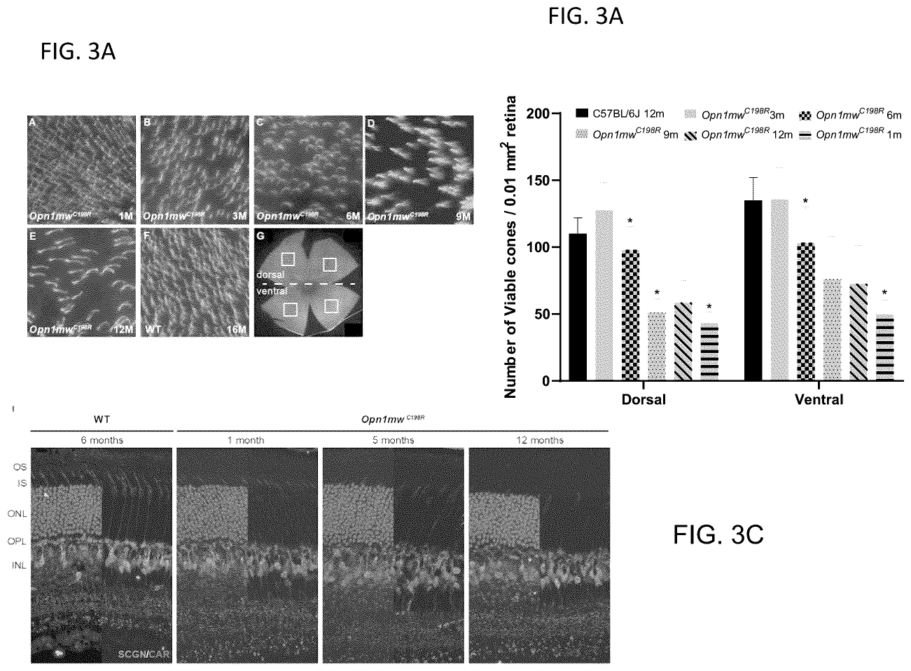

FIG. 3A to FIG. 11 show characterization and gene therapy of Opn1mw^{C198R}Opn1sw^{-/-} mice.

FIG. 3A shows representative retinal whole mounts stained with Peanut agglutinin (PNA) to characterize cone photoreceptor degeneration. FIG. 3B shows the numbers of PNA positive cells were counted from dorsal and ventral areas of Opn1mw^{C198R}Opn1sw^{-/-} and wild-type mice. Opn1mw^{C198R}Opn1sw^{-/-} mice have similar numbers of viable cones as wildtype mice at 1 month of age, but cones degenerate between 1 and 6 months and stabilize by 6 months of age. N=6 mice (3 Females and 3 males) were used for each group. 4 images (2 from dorsal and 2 from ventral) were taken from each retinas. P<0.05. FIG. 3C shows cone arrestin (red) and Secretagogin (green) staining to characterize cones and cone bipolar in aged Opn1mw^{C198R}Opn1sw^{-/-} mice. Cone arrestin staining showed that cones degenerate with age and cone outer segments are absent in Opn1mw^{C198R}Opn1sw^{-/-} mice. Secretagogin staining showed that cone bipolar cells appeared to be normal up to 12 months of age.

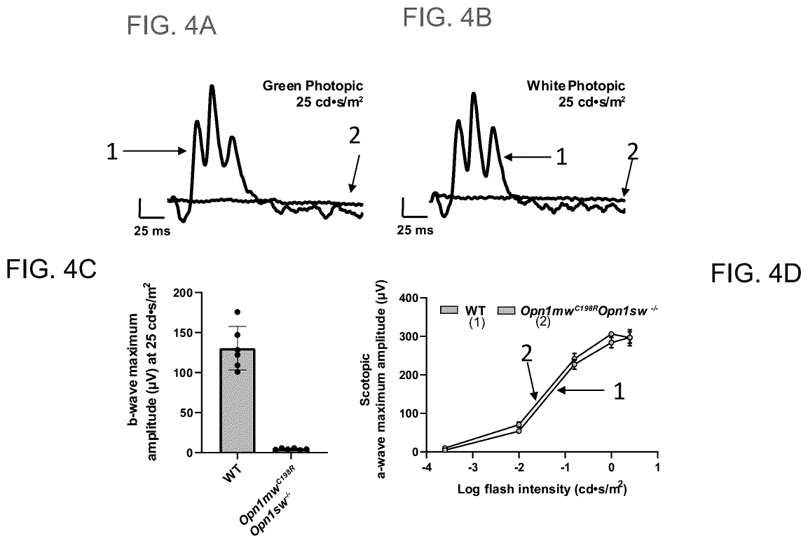

FIG. 4A shows that Opn1mw^{C198R}Opn1sw^{-/-} mice have abolished photopic electroretinal responses (ERG) while maintain normal scotopic ERG; representative middle-wavelength mediated ERG traces from wild-type (grey (1)) and Opn1mw^{C198R}Opn1sw^{-/-} (red (2)) mice. FIG. 4B shows representative white light mediated ERG traces from wild-type (grey (1)) and Opn1mw^{C198R}Opn1sw^{-/-} (red (2)) mice. FIG. 4C shows averaged white light b-wave maximum amplitudes at light intensity of 25 cd·s/m² from wild-type and Opn1mw^{C198R}Opn1sw^{-/-} mice. FIG. 4D shows averaged scotopic a-wave maximum amplitudes at different intensities to show that rod function is normal in Opn1mw^{C198R}Opn1sw-/-mice.

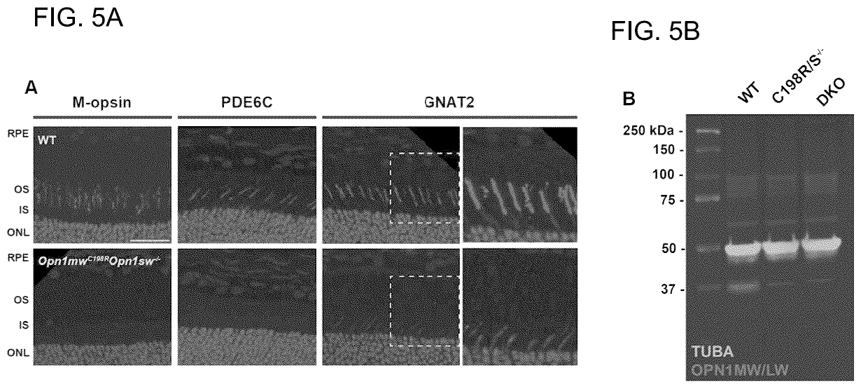

FIG. 5A shows that cone outer segments are absent in Opn1mw^{C198R}Opn1sw^{-/-} mice. Cone outer segments were stained with cone phosphodiesterase α′ (PDE6C) and GNAT2 in Opn1mw^{C198R}Opn1sw^{-/-} and wildtype mice. PDE6α′ and GNAT2 stainings are absent in Opn1mw^{C198R}Opn1sw^{-/-} retinas. FIG. 5B shows a Western blot analysis. The Western blot shows that expression of Opn1mw^{C198R} mutant opsin was not detected. The faint band near 37 KD was a non-specific band also present in the Opn1mw^{-/-}Opn1sw^{-/-} retinas (labeled as DKO) which have abolished expression of OPN1MW.

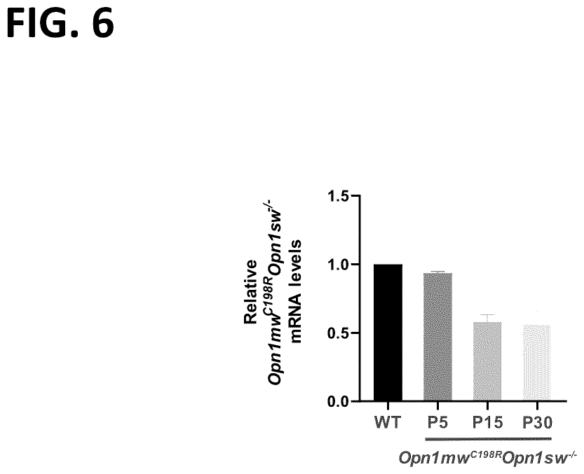

FIG. 6 shows real-time PCR results that mRNA levels of Opn1mw^{C198R} mutant in Opn1mw^{C198R}Opn1sw^{-/-} retinas are normal at postal natal 5 (P5) but only ˜50% of wild-type levels at P15 and P30.

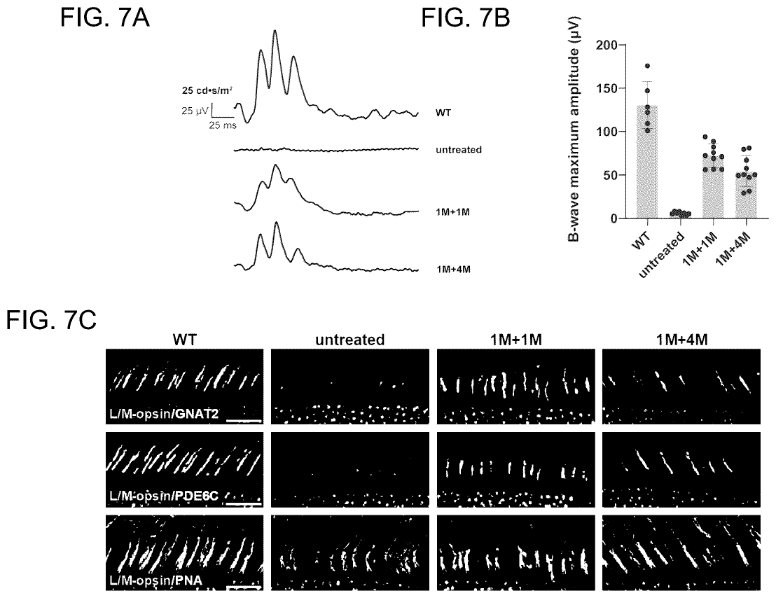

FIG. 7A shows gene therapy rescued cone function and structure in Opn1mw^{C198R} Opn1sw^{-/-} mice when treated at 1 month of age and shows representative cone-mediated medium-wavelength ERG traces from wild-type (black waveforms (1)), untreated Opn1mw^{C198R} Opn1sw^{-/-} (red waveforms (2)) and AAV5-hOPN1MW-treated at 1 month of age and ERGed at 1month (blue waveforms (3)) and 4 months (green waveforms (4)) post-injection. FIG. 7B shows averaged b-wave maximum amplitudes of middle-wavelength mediated ERG responses in Opn1mw^{C198R} Opn1sw^{-/-} untreated, wild-type controls, and Opn1mw^{C198R} Opn1sw^{-/-} treated with AAV5-hOPN1MW at 1 month post-injection (1M+1M) and 4 months post-injection (1M+4M). FIG. 7C shows immunohistochemistry shows that treatment restored expression and normal localization of cone outer segment specific proteins of GNAT2 and PDC6C.

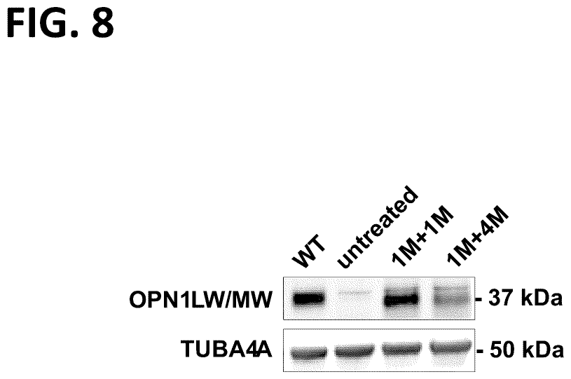

FIG. 8 shows western blot analysis OPN1M/LW expression in treated Opn1mw^{C198R} Opn1sw^{-/-} eyes injected at 1 month of age and analyzed 1 month and 4 months post-injection. An TUBA 4A antibody was used as loading control.

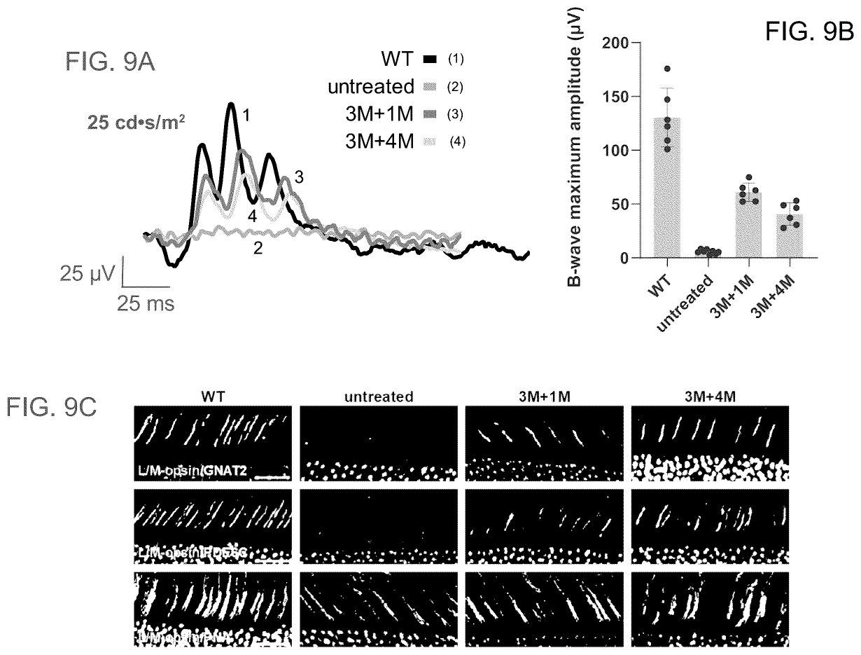

FIG. 9A shows gene therapy rescued cone function and structure in Opn1mw^{C198R} Opn1sw-mice when treated at 3 months of age and shows representative cone-mediated medium-wavelength ERG traces from wild-type (black waveforms (1)), untreated Opn1mw^{C198R} Opn1sw^{-/-} (red waveforms (2)) and AAV5-hOPN1MW-treated at 3 month of age and ERGed at 1 month (blue waveforms (3)) and 4 months (green waveforms (4)) post-injection. FIG. 9B shows averaged b-wave maximum amplitudes of middle-wavelength mediated ERG responses in Opn1mw^{C198R} Opn1sw^{-/-} untreated, wild-type controls, and Opn1mw^{C198R} Opn1sw^{-/-} treated with AAV5-hOPN1MW at 1 month post-injection (3M+1M) and 4 months post-injection (3M+4M). FIG. 9C shows immunohistochemistry shows that treatment restored expression and normal localization of cone outer segment specific proteins of GNAT2 and PDC6C.

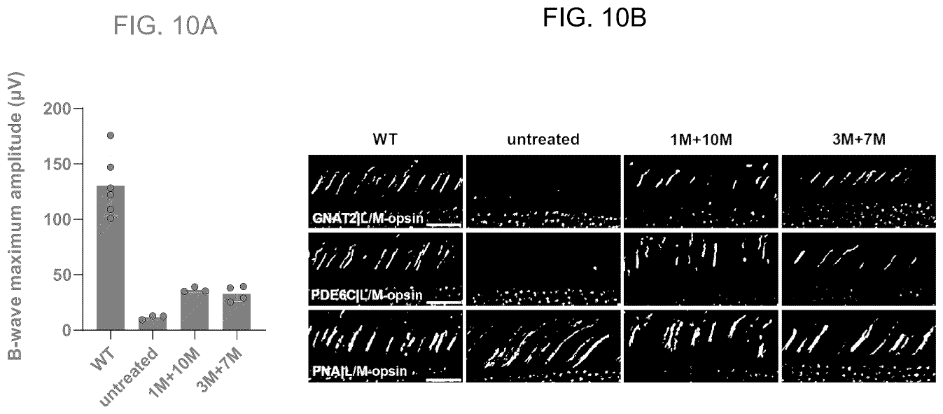

FIG. 10A shows cone-mediated function was maintained for long-term in Opn1mw^{C198R} Opn1sw^{-/-} mice treated at 1 and 3 months and shows averaged b-wave maximum amplitudes of middle-wavelength mediated ERG responses in Opn1mw^{C198R} Opn1sw^{-/-} untreated, wild-type controls, and Opn1mw^{C198R} Opn1sw^{-/-} treated at 1 month and ERGed 10 month post-injection (1M+10M), and treated at 3 month and ERGed 7 month post-injection (3M+7M). FIG. 10B shows immunohistochemistry shows that treatment restored expression and normal localization of cone outer segment specific proteins of GNAT2 and PDC6C.

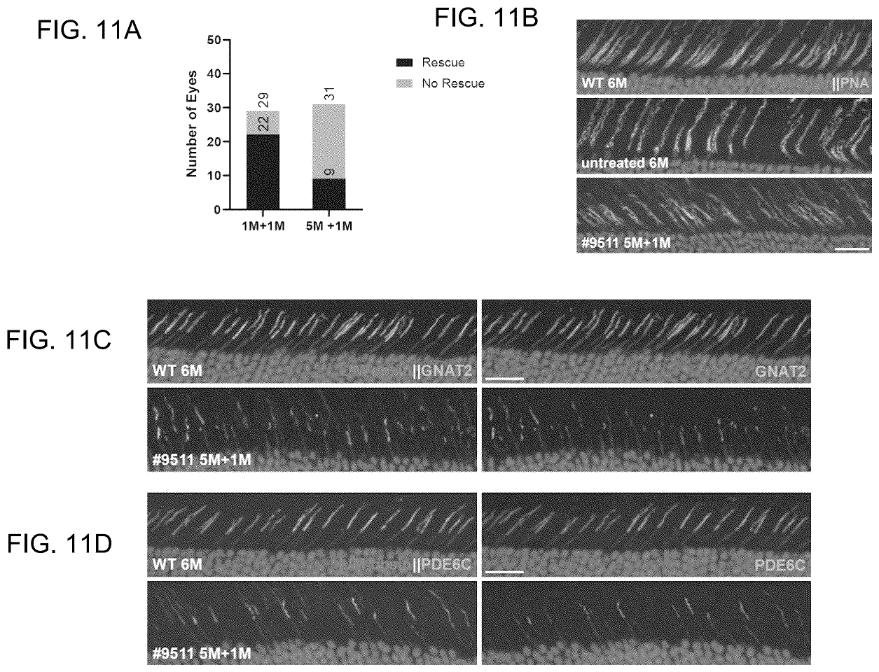

FIG. 11A shows that treatment efficacy was significantly diminished when Opn1mw^{C198R} Opn1sw^{-/-} mice were treated at 5 months of age and shows only ˜29% (9 out of 31 mice) of eyes treated at 5 months of age showed any cone rescue above 20 μV, while ˜75% of eyes (22 out of 29) treated at 1 months of age showed cone rescue above 50 μV. FIG. 11B shows immunohistochemistry shows that although a lot more cones are present by PNA staining, however, only 30-50% of PNA positive cones showed OPN1M/LW staining. FIG. 11C shows in 5M+1M treated eyes, only ˜50% of OPN1M/LW positive cells showed GNAT2 expression and the staining is much weaker compared to mice treated at 1 month and 3 month. FIG. 11D shows in 5M+1M treated eyes, only ˜50% of OPN1M/LW positive cells showed PDE6C expression and the staining is weaker compare to mice treated at 1 month and 3 month.

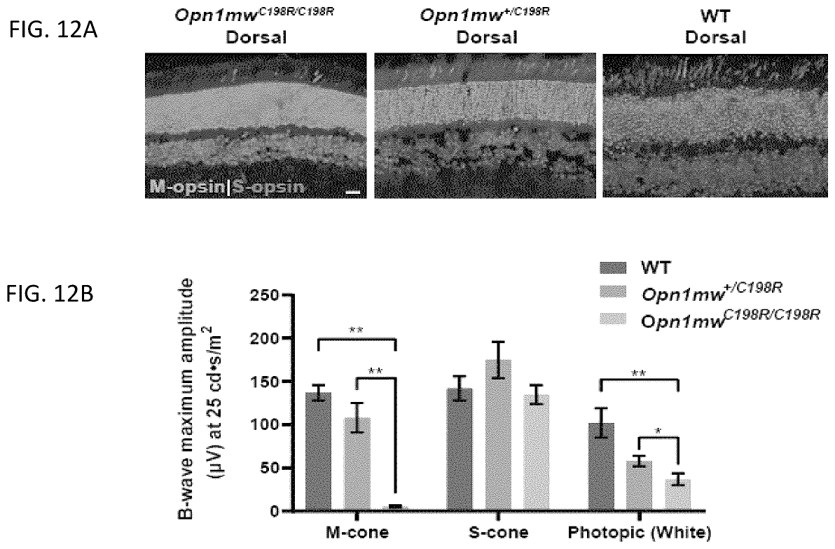

FIG. 12A to FIG. 18 show characterization and gene therapy of Opn1mw^{C198R} mice.

FIG. 12A shows in Opn1mw^{C198R} homozygous female mice, misfolded Opn1mw^{C198R} protein was not detected by immunohistochemistry. While in Opn1mw^{C198R} heterozygous female, the number of M-opsin positive cells are about half of in the wild-type mice. M-opsin was labeled as red and S-opsin was labeled as green. FIG. 12B shows in Opn1mw^{C198R} mice, there is no M-cone ERG in homozygous Females and hemizygous males, and M-cone ERG is reduced in heterozygous females. The S-cone ERG function is normal in Opn1mw^{C198R} mice. The photopic ERG is also reduced in Opn1mw^{C198R} mice most likely due to loss of S-cone function.

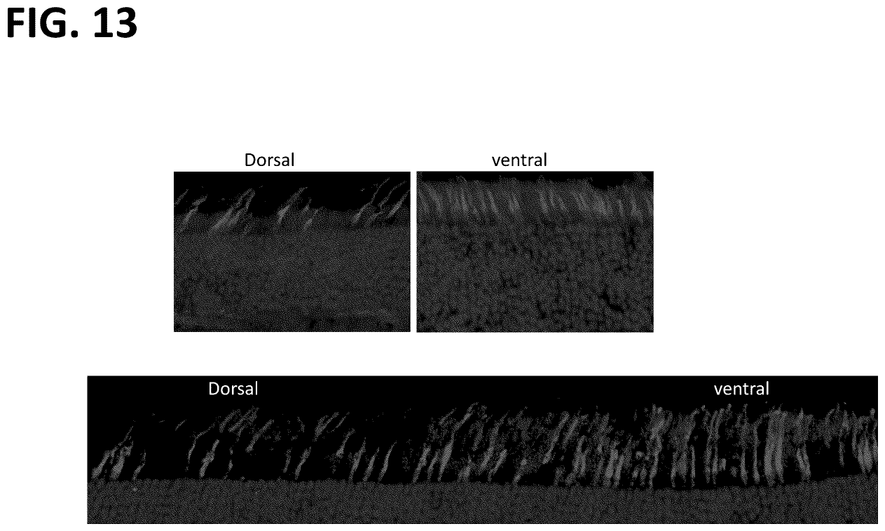

FIG. 13 shows on the top row dorsal and ventral retinas and bottom row shows that cones degenerate in the dorsal retinas of 6 months old Opn1mw^{C198R} mice. Cones were labeled with PNA. There were less PNA positive cells in the dorsal retinas than in the ventral area suggesting cones die gradually with age.

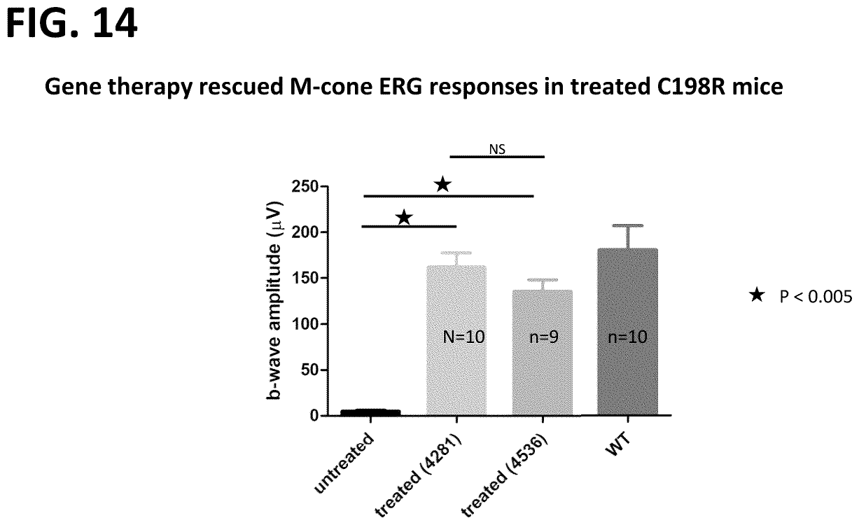

FIG. 14 shows averaged b-wave maximum amplitudes of middle-wavelength mediated ERG responses in Opn1mw^{C198R} untreated mice, Opn1mw^{C198R} mice treated with either AAV5-PR2.1-hOPN1MW.HA or AAV5-PR2.1-hOPN1LW, and isogenic wild-type controls. Opn1mw^{C198R} mice were treated at 1 month of age and ERGed at 1 month post-injection.

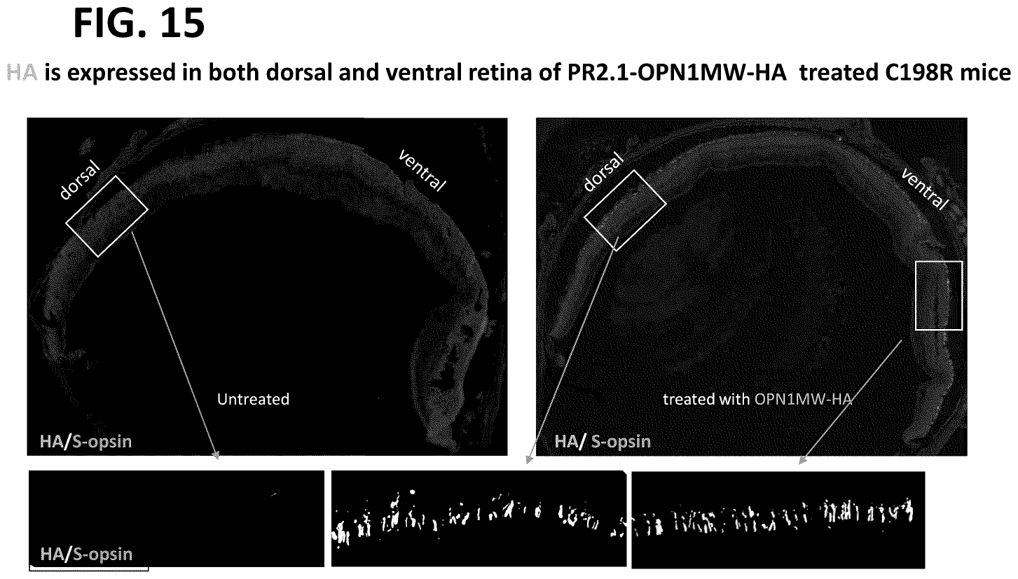

FIG. 15 top row (left) and bottom row (left) shows untreated dorsal retina, and top row (right) shows dorsal and ventral retinas treated with AAV-mediated hOPN1MW.HA expression in cone outer segments in both dorsal (bottom middle FIG. 15) and ventral (bottom right FIG. 15) areas of treated Opn1mw^{C198R} mice. HA tag was included in frame at the C-terminal of hOPN1MW.

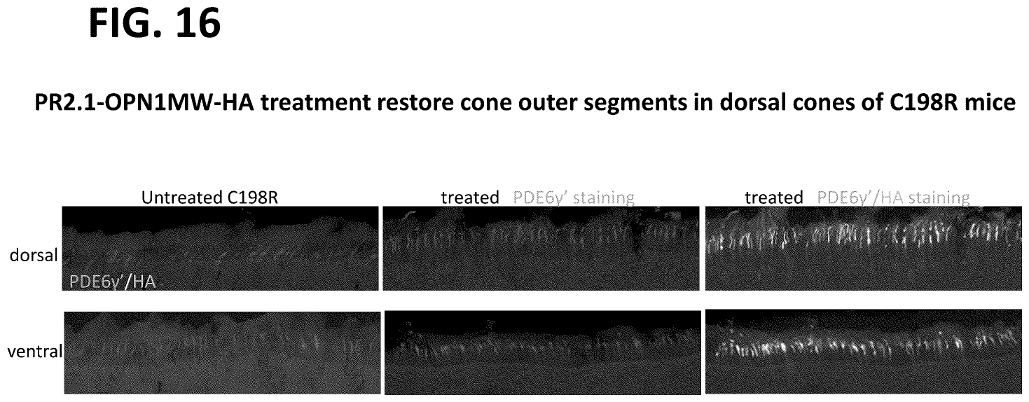

FIG. 16 shows AAV5-PR2.1-hOPN1MW.HA gene therapy restored cone phosphodiesterase γ′ subunit (PDE6 γ′) expression and subcellular localization in the dorsal retinas of treated Opn1mw^{C198R} mice. In the dorsal retinas of untreated Opn1mw^{C198R} mice, cone outer segments were significantly shortened, and no PDE6 γ′ expression was detected. In contrast, in the AAV5-PR2.1-hOPN1MW.HA treated dorsal retinas, treatment restored normal cone outer segments and PDE6 γ′ expression and localization.

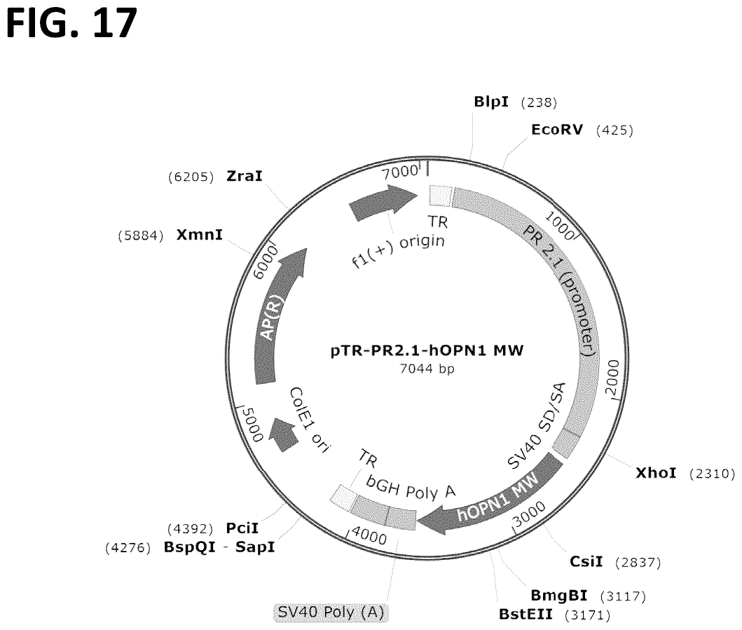

FIG. 17 shows maps of an illustrative vector of a certain embodiment of this invention containing the human OPN1MW cDNA driven by cone-specific PR2.1 promoter.

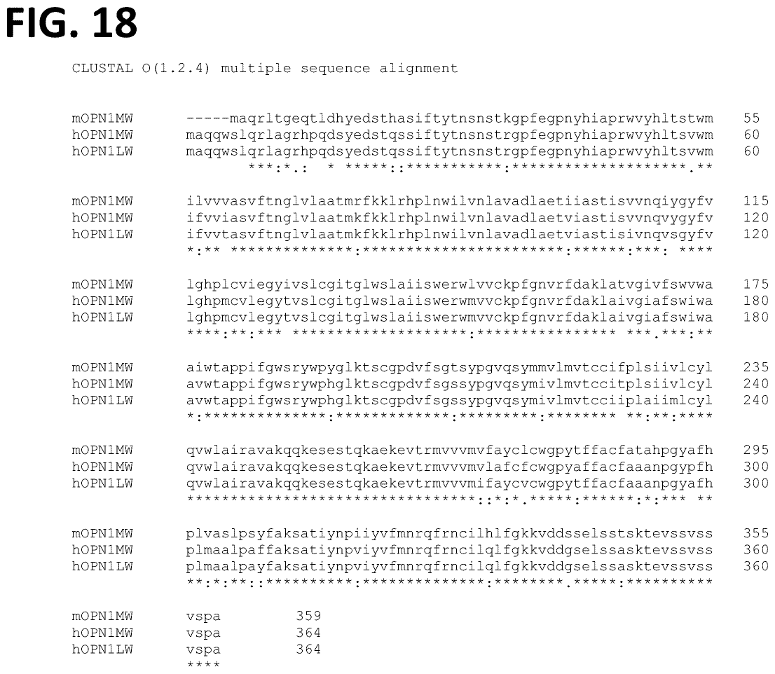

FIG. 18 shows amino acid sequence alignment of mouse OPN1MW (i.e. mOPN1MW), human OPN1MW (i.e. hOPN1MW), and human OPN1LW (i.e. hOPN1LW). Protein sequences are highly conserved between mouse and human opsins.

In certain embodiments of this invention, a composition comprising rAAV vectors that express a biologically-functional cone opsin peptide, polypeptide, or protein, and a pharmaceutically acceptable carrier, are provided. The composition includes wherein said rAAV vector expresses either rAAV-OPN1LW or rAAV-OPN1MW.

In other embodiments of this invention, a method is provided for treating a patient having an eye disease, disorder, trauma, injury, or dysfunction comprising administering to a patient a therapeutically effective amount of a composition comprising a rAAV vector that express a biologically-functional cone opsin peptide, polypeptide, or protein, and a pharmaceutically acceptable carrier. This method includes wherein said rAAV vector is either rAAV-OPN1LW or rAAV-OPN1MW. The method may include wherein said disorder or disease is of a mammalian eye, and is a congenital retinal blindness. This method includes wherein said congentital retinal blindness is selected from the group of retinal dystrophy such as cone opsin deficiency and blue cone monochromacy (BCM) in humans caused by mis-sense point mutations, such as C203R, in the OPN1LW/OPN1MW.

In yet another embodiment of this invention, a method is provided for preparing a rAAV vector-based composition for use in viral vector-based gene therapies, including, preparing a rAAV-OPN1LW vector or a rAAV-OPN1MW vector driven by a cone specific promoter PR2.1.

Another embodiment of this invention provides a method of treating a patient having blue cone monochromacy comprising administering to a patient a therapeutically effective amount of a composition gene replacement using rAAV vectors that encode one or more mammalian cone opsins polypeptides for treating a cone photoreceptor function of the patent. This method includes wherein said rAAV vector expresses either rAAV-OPN1LW or rAAV-OPN1MW. This method includes wherein said rAAV vector expresses either rAAV-OPN1LW or rAAV-OPN1MW driven by a cone specific promoter PR2.1.

In certain embodiments of this invention, a viral vector is provided comprising a vector containing a human OPN1MWcDNA driven by cone-specific PR2.1 promoter12.

In certain embodiments of this invention, a vector containing a human OPN1MWcDNA driven by cone-specific PR2.1 promoter is provided.

In another embodiment of this invention, a method is disclosed of treating a patient carrying a C203R mutation wherein cysteine in protein position 203 is mutated to arginine, comprising administering to a patient a therapeutically effective amount of a recombinant adeno-associated viral vector expressing either human L-opsin or M-opsin driven by a cone photoreceptor-cell specific promoter. This method includes wherein said administration of said composition is by injecting said composition into an eye of said patient.

A SEQ ID NO:1 of mouse OPN1MW (i.e. mOPN1MW) is provided.

A SEQ ID NO:2 of human OPN1MW (i.e. hOPN1MW) and a SEQ ID NO:3 of human OPN1LW (i.e. hOPN1LW) are provided.

SEQ ID NO: 1
mOPN1MW -----maqrltgeqtldhyedsthasiftytnsnstkgpfegpnyhiaprwvyhltstwm 55
mOPN1MW ilvvvasvftnglvlaatmrfkklrhplnwilvnlavadlaetiiastisvvnqiygyfv 115
mOPN1MW lghplcviegyivslcgitglwslaiiswerwlvvckpfgnvrfdaklatvgivfswvwa 175
mOPN1MW aiwtappifgwsrywpyglktscgpdvfsgtsypgvqsymmvlmvtccifplsiivlcyl 235
mOPN1MW qvwlairavakqqkesestqkaekevtrmvvvmvfayclcwgpytffacfatahpgyafh 295
mOPN1MW plvaslpsyfaksatiynpiiyvfmnrqfrncilhlfgkkvddsselsstsktevssvss 355
mOPN1MW vspa 359
hOPN1MW vspa 364
SEQ ID NO: 2
hOPN1MW maqqwslqrlagrhpqdsyedstqssiftytnsnstrgpfegpnyhiaprwvyhltsvwm 60
hOPN1MW ifvviasvftnglvlaatmkfkklrhplnwilvnlavadlaetviastisvvnqvygyfv 120
hOPN1MW lghpmcvlegytvslcgitglwslaiiswerwmvvckpfgnvrfdaklaivgiafswiwa 180
hOPN1MW qvwlairavakqqkesestqkaekevtrmvvvmvlafcfcwgpyaffacfaaanpgypfh 300
hOPN1MW plmaalpaffaksatiynpviyvfmnrqfrncilqlfgkkvddgselssasktevssvss 360
hOPN1MW vspa 364
SEQ ID NO: 3
hOPN1LW maqqwslqrlagrhpqdsyedstqssiftytnsnstrgpfegpnyhiaprwvyhltsvwm 60
hOPN1LW ifvvtasvftnglvlaatmkfkklrhplnwilvnlavadlaetviastisivnqvsgyfv 120
hOPN1LW lghpmcvlegytvslcgitglwslaiiswerwmvvckpfgnvrfdaklaivgiafswiwa 180
hOPN1LW avwtappifgwsrywphglktscgpdvfsgssypgvqsymivlmvtcciiplaiimlcyl 240
hOPN1LW qvwlairavakqqkesestqkaekevtrmvvvmifaycvcwgpytffacfaaanpgyafh 300
hOPN1LW plmaalpayfaksatiynpviyvfmnrqfrncilqlfgkkvddgselssasktevssvss 360
hOPN1LW vspa 364

### REFERENCES

1. Curcio CA, Allen KA, Sloan KR et al. Distribution and morphology of human cone photoreceptors stained with anti-blue opsin. J Comp Neurol 1991;312:610-624.
2. Curcio CA, Sloan KR, Kalina RE et al. Human photoreceptor topography. J Comp Neurol 1990;292:497-523.
3. Mustafi D, Engel AH, Palczewski K. Structure of cone photoreceptors. Prog Retin Eye Res 2009;28:289-302.
4. Nathans J, Thomas D, Hogness DS. Molecular genetics of human color vision: the genes encoding blue, green, and red pigments. Science 1986;232:193-202.
5. Ayyagari R, Kakuk LE, Coats CL et al. Bilateral macular atrophy in blue cone monochromacy (BCM) with loss of the locus control region (LCR) and part of the red pigment gene. Mol Vis 1999;5:13.
6. Carroll J, Baraas RC, Wagner-Schuman M et al. Cone photoreceptor mosaic disruption associated with Cys203 Arg mutation in the M-cone opsin. Proc Natl Acad Sci U S A 2009;106:20948-20953.
7. Carroll J, Neitz M, Hofer H et al. Functional photoreceptor loss revealed with adaptive optics: an alternate cause of color blindness. Proc Natl Acad Sci U S A 2004;101:8461-8466.
8. Crognale MA, Fry M, Highsmith J et al. Characterization of a novel form of X-linked incomplete achromatopsia. Vis Neurosci 2004;21:197-203.
9. Gardner JC, Michaelides M, Holder GE et al. Blue cone monochromacy: causative mutations and associated phenotypes. Mol Vis 2009;15:876-884.
10. Gardner JC, Webb TR, Kanuga N et al. X-linked cone dystrophy caused by mutation of the red and green cone opsins. Am J Hum Genet 2010;87:26-39.
11. Jagla WM, Jagle H, Hayashi T et al. The molecular basis of dichromatic color vision in males with multiple red and green visual pigment genes. Hum Mol Genet 2002;11:23-32.
12. Kellner U, Wissinger B, Tippmann S et al. Blue cone monochromatism: clinical findings in patients with mutations in the red/green opsin gene cluster. Graefes Arch Clin Exp Ophthalmol 2004;242:729-735.
13. Michaelides M, Johnson S, Bradshaw K et al. X-linked cone dysfunction syndrome with myopia and protanopia. Ophthalmology 2005;112:1448-1454.
14. Michaelides M, Johnson S, Simunovic MP et al. Blue cone monochromatism: a phenotype and genotype assessment with evidence of progressive loss of cone function in older individuals. Eye (Lond) 2005;19:2-10.
15. Mizrahi-Meissonnier L, Merin S, Banin E et al. Variable retinal phenotypes caused by mutations in the X-linked photopigment gene array. Invest Ophthalmol Vis Sci 2010;51:3884-3892.
16. Nathans J, Davenport CM, Maumenee IH et al. Molecular genetics of human blue cone monochromacy. Science 1989;245:831-838.
17. Nathans J, Maumenee IH, Zrenner E et al. Genetic heterogeneity among blue-cone monochromats. Am J Hum Genet 1993;53:987-1000.
18. Neitz M, Carroll J, Renner A et al. Variety of genotypes in males diagnosed as dichromatic on a conventional clinical anomaloscope. Vis Neurosci 2004;21:205-216.
19. Winderickx J, Sanocki E, Lindsey DT et al. Defective colour vision associated with a missense mutation in the human green visual pigment gene. Nat Genet 1992;1:251-256.
20. Vollrath D, Nathans J, Davis RW. Tandem array of human visual pigment genes at Xq28.Science 1988;240:1669-1672.
21. Smallwood PM, Wang Y, Nathans J. Role of a locus control region in the mutually exclusive expression of human red and green cone pigment genes. Proc Natl Acad Sci U S A 2002;99:1008-1011.
22. Wang Y, Macke JP, Merbs SL et al. A locus control region adjacent to the human red and green visual pigment genes. Neuron 1992;9:429-440.
23. Yamaguchi T, Motulsky AG, Deeb SS. Visual pigment gene structure and expression in human retinae. Hum Mol Genet 1997;6:981-990.
24. Winderickx J, Battisti L, Motulsky AG et al. Selective expression of human X chromosome-linked green opsin genes. Proc Natl Acad Sci U S A 1992;89:9710-9714.
25. Neitz J, Neitz M. The genetics of normal and defective color vision. Vision Res 2011;51:633-651.
26. Reyniers E, Van Thienen MN, Meire F et al. Gene conversion between red and defective green opsin gene in blue cone monochromacy. Genomics 1995;29:323-328.
27. Kazmi MA, Sakmar TP, Ostrer H. Mutation of a conserved cysteine in the X-linked cone opsins causes color vision deficiencies by disrupting protein folding and stability. Invest Ophthalmol Vis Sci 1997;38:1074-1081.
28. Buena-Atienza E, Ruther K, Baumann B et al. De novo intrachromosomal gene conversion from OPN1MW to OPNILW in the male germline results in Blue Cone Monochromacy. Sci Rep 2016;6:28253.
29. Kohl S, Jagle H, Wissinger B. Achromatopsia. In: GeneReviews (R). RA Pagon, MP Adam, HH Ardingeret al., eds. (Seattle (WA)). 1993.
30. Michaelides M, Hunt DM, Moore AT. The cone dysfunction syndromes. Br J Ophthalmol 2004;88:291-297.
31. Richards JE, Scott KM, Sieving PA. Disruption of conserved rhodopsin disulfide bond by Cys187Tyr mutation causes early and severe autosomal dominant retinitis pigmentosa. Ophthalmology 1995;102:669-677.
32. Berson EL, Sandberg MA, Rosner B et al. Color plates to help identify patients with blue cone monochromatism. Am J Ophthalmol 1983;95:741-747.
33. Hess RF, Mullen KT, Sharpe LT et al. The photoreceptors in atypical achromatopsia. J Physiol 1989;417:123-149.
34. Carroll J, Dubra A, Gardner JC et al. The effect of cone opsin mutations on retinal structure and the integrity of the photoreceptor mosaic. Invest Ophthalmol Vis Sci 2012;53:8006-8015.
35. Cideciyan AV, Hufnagel RB, Carroll J et al. Human cone visual pigment deletions spare sufficient photoreceptors to warrant gene therapy. Hum Gene Ther 2013;24:993-1006.
36. Carroll J, Rossi EA, Porter J et al. Deletion of the X-linked opsin gene array locus control region (LCR) results in disruption of the cone mosaic. Vision Res 2010;50:1989-1999.
37. Zhang Y, Deng WT, Du W et al. Gene-based Therapy in a Mouse Model of Blue Cone Monochromacy. Sci Rep 2017;7:6690.
38. Deng WT, Li J, Zhu P et al. Human L-and M-opsins restore M-cone function in a mouse model for human blue cone monochromacy. Mol Vis 2018;24:17-28.
39. Ma X, Sechrest ER, Fajardo D et al. Gene Therapy in Opn1mw(−/−)/Opn1sw(−/−) Mice and Implications for Blue Cone Monochromacy Patients with Deletion Mutations. Hum Gene Ther 2022;33:708-718.
40. Zhu P, Dyka F, Ma X et al. Disease mechanisms of X-linked cone dystrophy caused by missense mutations in the red and green cone opsins. FASEB J 2021;35:e21927

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

It is further to be understood that all values are approximate, and are provided for description.

All patents, applications, publications, test methods, literature, and other materials cited herein are incorporated by reference. If there is a discrepancy between (a) the incorporated by reference patents, applications, publications, test methods, literature, and other materials, and (b) the present application, then the present application's specification, figures, and claims control the meaning of any terms and the scope of the inventions set forth herein.

1. A composition comprising rAAV vectors that express a biologically-functional cone opsin peptide, polypeptide, or protein, and a pharmaceutically acceptable carrier.

2. The composition of claim 1 wherein said rAAV vector expresses either rAAV-OPN1LW or rAAV-OPN1MW.

3. A method of treating a patient having an eye disease, disorder, trauma, injury, or dysfunction comprising administering to a patient a therapeutically effective amount of a composition of claim 1.

4. The method of claim 3 including wherein said rAAV vector is either rAAV-OPN1LW or rAAV-OPN1MW.

5. The method of claim 3 wherein said disorder or disease is of a mammalian eye, and is a congenital retinal blindness.

6. The method of claim 5 wherein said congenital retinal blindness is selected from the group of retinal dystrophy such as cone opsin deficiency and blue cone monochromacy (BCM) in humans caused by mis-sense point mutations, such as C203R, in the OPN1LW/OPN1MW.

7. The method of claim 3, wherein the rAAV-OPN1LW vector or a rAAV-OPN1MW vector is driven by a cone specific promoter PR2.1.

8. A method of treating a patient having blue cone monochromacy comprising administering to a patient a therapeutically effective amount of a composition gene replacement using rAAV vectors that encode one or more mammalian cone opsins polypeptides for treating a cone photoreceptor function of the patent.

9. The method of claim 8 including wherein said rAAV vector expresses either rAAV-OPN1LW or rAAV-OPN1MW.

10. The method of claim 9 including wherein said rAAV vector expresses either rAAV-OPN1LW or rAAV-OPN1MW driven by a cone specific promoter PR2.1.

11. A viral vector selected from the group consisting of a vector containing a human OPN1MWcDNA driven by cone-specific PR2.1 promoter12 and a vector containing a human OPN1MWcDNA driven by cone-specific PR2.1 promoter.

12. The viral vector of claim 11 having a vector map:

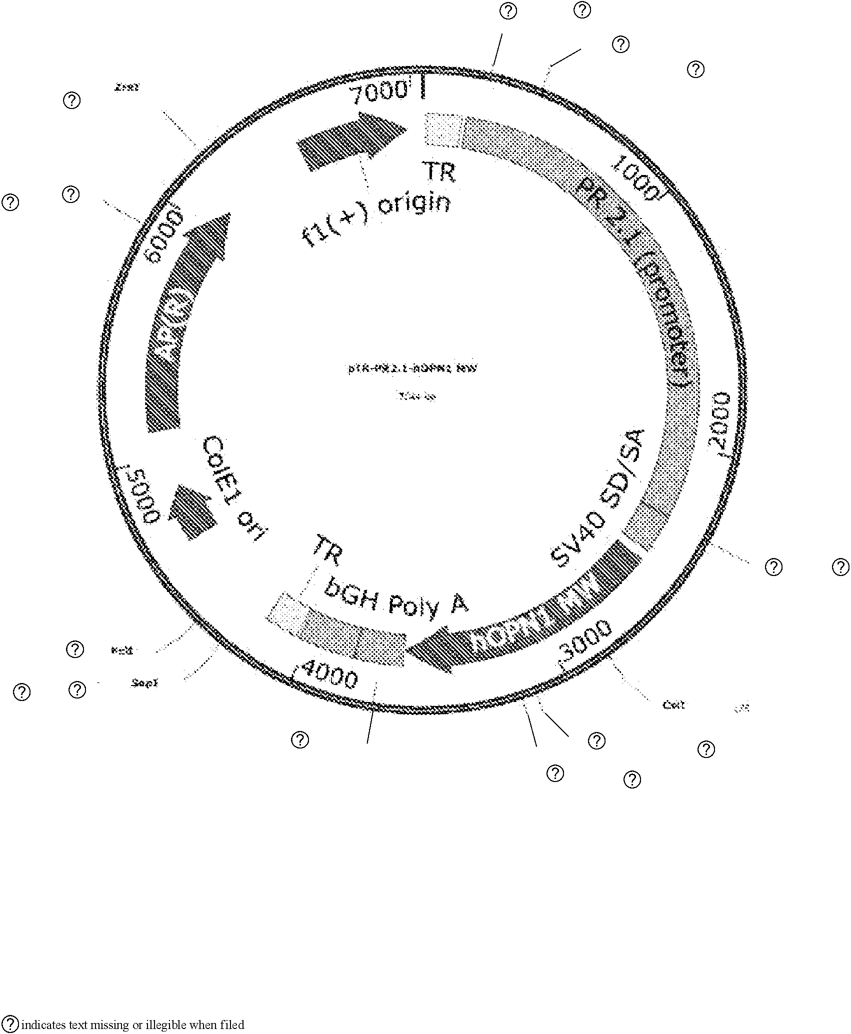

13. (canceled)

14. A method of treating a patient carrying a C203R mutation wherein cysteine in protein position 203 is mutated to arginine, comprising administering to a patient a therapeutically effective amount of a recombinant adeno-associated viral vector expressing either human L-opsin or M-opsin driven by a cone photoreceptor-cell specific promoter.

15. The method of claim 14 including wherein said administration of said composition is by injecting said composition into an eye of said patient.

16. An amino acid sequence selected from the group consisting of A SEQ ID NO:2 and SEQ ID NO: 3.

17. The SEQ ID NO:2 of claim 16 that is of hOPN1MW.

18. (canceled)

19. The SEQ ID NO:3 of claim 16 that is of hOPN1LW.


SEQUENCE LISTING (3 sequences)

SEQ ID NO: 1 - protein, 363 aa, synthetic construct
  REGION 1..359: note = mouse M-opsin (mOPN1MW) amino acid sequence
  REGION 360..363: note = human M-opsin (hOPN1MW) amino acid sequence
MAQRLTGEQTLDHYEDSTHASIFTYTNSNSTKGPFEGPNYHIAPRWVYHLTSTWMILVVVASVFTNGLVLAATMRFKKLRHPLNWILVNLAVADLAETIIASTISVVNQIYGYFVLGHPLCVIEGYIVSLCGITGLWSLAIISWERWLVVCKPFGNVRFDAKLATVGIVFSWVWAAIWTAPPIFGWSRYWPYGLKTSCGPDVFSGTSYPGVQSYMMVLMVTCCIFPLSIIVLCYLQVWLAIRAVAKQQKESESTQKAEKEVTRMVVVMVFAYCLCWGPYTFFACFATAHPGYAFHPLVASLPSYFAKSATIYNPIIYVFMNRQFRNCILHLFGKKVDDSSELSSTSKTEVSSVSSVSPAVSPA

SEQ ID NO: 2 - protein, 304 aa, synthetic construct
  REGION 1..304: note = human M-opsin (hOPN1MW) amino acid sequence
MAQQWSLQRLAGRHPQDSYEDSTQSSIFTYTNSNSTRGPFEGPNYHIAPRWVYHLTSVWMIFVVIASVFTNGLVLAATMKFKKLRHPLNWILVNLAVADLAETVIASTISVVNQVYGYFVLGHPMCVLEGYTVSLCGITGLWSLAIISWERWMVVCKPFGNVRFDAKLAIVGIAFSWIWAQVWLAIRAVAKQQKESESTQKAEKEVTRMVVVMVLAFCFCWGPYAFFACFAAANPGYPFHPLMAALPAFFAKSATIYNPVIYVFMNRQFRNCILQLFGKKVDDGSELSSASKTEVSSVSSVSPA

SEQ ID NO: 3 - protein, 364 aa, synthetic construct
  REGION 1..364: note = human M-opsin (hOPN1LW) amino acid sequence
MAQQWSLQRLAGRHPQDSYEDSTQSSIFTYTNSNSTRGPFEGPNYHIAPRWVYHLTSVWMIFVVTASVFTNGLVLAATMKFKKLRHPLNWILVNLAVADLAETVIASTISIVNQVSGYFVLGHPMCVLEGYTVSLCGITGLWSLAIISWERWMVVCKPFGNVRFDAKLAIVGIAFSWIWAAVWTAPPIFGWSRYWPHGLKTSCGPDVFSGSSYPGVQSYMIVLMVTCCIIPLAIIMLCYLQVWLAIRAVAKQQKESESTQKAEKEVTRMVVVMIFAYCVCWGPYTFFACFAAANPGYAFHPLMAALPAYFAKSATIYNPVIYVFMNRQFRNCILQLFGKKVDDGSELSSASKTEVSSVSSVSPA